(19) United States
(12) Patent Application Publication
(10) Pub. No.: US 2004/0192958 A1
(43) Pub. Date: Sep. 30, 2004

(54) Process for preparing derivatives of 4-halobutyraldehyde

(76) Inventor: John Anthony Hyatt, Kingsport, TN (US)

Correspondence Address: Michael J. Blake, Eastman Chemical Company, P.O. Box 511, Kingsport, TN, 37662-5075, US

(21) Appl. No.: 10/395,994

(22) Filed: Mar. 25, 2003

(51) Int. Cl. C07C 67/08

(52) U.S. Cl. 560/238

(57) ABSTRACT

The present invention describes a process for preparing an enol ester of a 4-halo-butyraldehyde which comprises contacting a cyclopropane carboxaldehyde (CPCA) and a carboxylic acid halide in the presence of a Lewis acid catalyst. In addition, the present invention relates to a process for preparing an acetal of a 4-halo-butyraldehyde which comprises contacting an enol ester of a 4-halo-butyraldehyde, prepared according to the present invention, with an alcohol in the presence of a certain acid catalyst.

The instant invention is a process for preparing certain acetals corresponding to 4-halobutyraldehyde. In the process, cyclopropane carboxaldehyde is contacted with a carboxylic acid halide to form a 4-halo-1-buten-1-ol ester, an enol ester. The resulting enol ester is contacted with certain acids in the presence of an alcohol to form a corresponding acetal of 4-halobutyraldehyde.

### BACKGROUND OF THE INVENTION

Dimethyl and diethyl acetals of 4-chlorobutryaldehyde and 4-bromobutyraldehyde (i.e., 1,1-dimethoxy-4-chlorobutane, 1,1-diethoxy-4-chlorobutane, 1,1-diethoxy-4-bromobutane, and 1,1-dimethoxy-4-bromobutane) are known to be useful intermediates in the syntheses of commercially valuable pharmaceuticals, particularly those containing substituted indole rings. (See, e.g., J. A. Salmon et al., J. Med. Chem., 1995, 38(18), 3566; G. R. Martin et al., U.S. Pat. No. 5,466,699; Forbes et al., J. Chem. Soc., 1977, 2353; C. Chen, et al., J. Org. Chem., 1994, 59, 3738).

Such acetals have heretofore been prepared by the following synthetic route:

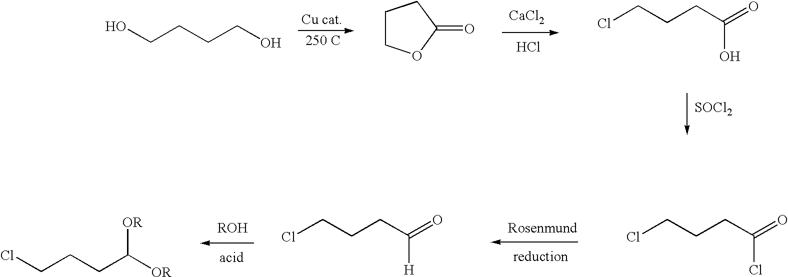

(see K. Weissermel and H. Arpe, Industrial Organic Chemistry, Verlag Chemie, NY 1978, p. 90; T. Athar, Indian J. Chem. (1998), 37B, 1037; Andrade et al., U.S. Pat. No. 4,691,062; I. Grandberg et al., Khim. Geterotsikl. Soedin 1968 (6), 1038; CA 70:68046). The foregoing 5-step sequence provides an overall yield of less than 22%.

Although others describe shorter routes to the intended acetals, such alternatives require the use of difficult, very-low-temperature reactions, such as a Swern oxidation, and are not suitable for industrial production. (See Crombie, et al., Tetrahedron Lett., 1985, 26(20), 2477). Similarly, other processes to obtain the acetals of interest have employed reagents such as pyridinium chlorochromate, which exhibits serious pollution problems (S. Kulkarni, Heterocycles, 1982 (18), 163).

### BRIEF SUMMARY OF THE INVENTION

The present invention describes a process for preparing an enol ester of a 4-halo-butyraldehyde which comprises contacting cyclopropane carboxaldehyde (CPCA) and a carboxylic acid halide having the formula R-COX in the presence of a Lewis acid catalyst, wherein R is as described below and X is halogen. In addition, the present invention also relates to a process for preparing an acetal of a 4-halo-butyraldehyde which comprises contacting an enol ester of a 4-halo-butyraldehyde, prepared according to the present invention, with an alcohol having the formula R¹OH in the presence of a certain acid catalyst, wherein R¹ is as described below.

The initial reaction above, in which a carboxylic acid halide and cyclopropane carboxaldehyde provide a 4-halo enol ester, as well as the overall reaction to produce an acetal of 4-halo-butyraldehyde, are both novel and unexpected based on the published literature.

### DETAILED DESCRIPTION OF THE INVENTION

According to the present invention, cyclopropane carboxaldehyde (CPCA) may be contacted with certain carboxylic acid halides in the presence of certain Lewis acid catalysts to produce 4-halo-1-carboxy-1-butenes, an enol ester, which may then be contacted with certain alcohols in the presence of acid catalysts provide 4-halobutyraldehyde acetals. According to the present process, the overall process is shorter than previously described technology, and the intended acetals are typically obtained in greater than 50% overall yield based on the CPCA starting material.

The instant process may be depicted in summary fashion by the following overall reaction scheme:

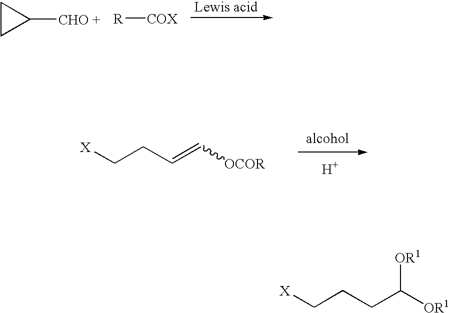

In the first step of the process of the present invention, the commercially available compound cyclopropane carboxaldehyde is contacted with a carboxylic acid halide in the presence of a Lewis acid catalyst. The product of this reaction is a mixture of E and Z isomers of an enol ester of 4-halobutryaldehyde, and may be depicted as follows:

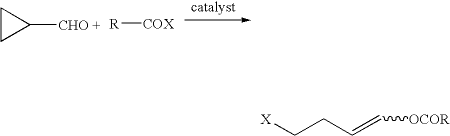

The carboxylic acid halide suitable for use in the process of the present invention is described by the general formula R-COX. In the foregoing formula, R is a straight or branched chain aliphatic hydrocarbon having 1 to 18 carbon atoms, a cyclic hydrocarbon having 3 to 8 carbon atoms, or an aromatic group having 6 to 18 carbon atoms. It is preferred that R be a straight or branched chain aliphatic hydrocarbon having 1 to 7 carbons. In a preferred embodiment of the present invention, R is methyl (one carbon) or ethyl (two carbons). In the carboxylic acid halide above, X represents a halogen atom (or a halide anion); preferably, X is chlorine or bromine (e.g., chloride or bromide). Among the preferred carboxylic acid halides for use in the present invention, acetyl chloride or acetyl bromide are particularly preferred.

The Lewis acid catalyst for use in the present invention may be any of the Lewis acids that are known and available to those of skill in the art. Preferred Lewis acids for use herein include zinc chloride, zinc bromide, zinc iodide, titanium tetrachloride, and titanium tetrabromide. More preferably, zinc chloride or zinc bromide are used. The amount of catalyst used can be in the range of about 0.05 to about 25 mole % with respect to the cyclopropane carboxaldehyde. In a preferred embodiment, the process of the instant invention employs an amount of Lewis acid catalyst in an amount of about 0.1 to about 10 mole % and, more preferably, employs an amount of catalyst in the range of about 0.5 to about 2.5 mole %.

An organic solvent can be used if desired, but is not necessary. Suitable solvents include halocarbons such as chloroform, carbon tetrachloride, tetrachloroethylene, and dichloromethane; esters such as methyl or ethyl acetate; ethers such as diethyl ether, diisopropyl ether, or tetrahydrofuran; straight or branched hydrocarbons having from 4 to 18 carbon atoms such as hexane, heptane or cyclohexane; or aromatics such as benzene, toluene, xylenes, or mesitylene, chlorobenzene, and the like. When a solvent is used, it is preferred to use ethyl acetate.

The reaction to produce the enol ester may be carried out over a wide range of temperatures, such as from about −20° to about +110° Centigrade. In a preferred embodiment the reaction is carried between about 20° and about 70° C., and more preferably between about 30° and about 50° C. The process may be carried out at about atmospheric, or ambient, pressure.

In the second step of the process of this invention, the enol ester prepared as described above may be contacted with an excess of an alcohol or a diol in the presence of an acid catalyst. In this step of the invention, the enol ester is converted into an acetal, which may be depicted according to the following equation:

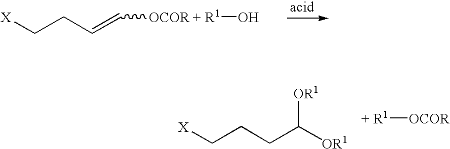

The alcohol for use in the second step of my invention may be a glycol or an alcohol according to the formula R¹OH. The moiety R¹ above may be chosen from any straight or branched chain or cyclic aliphatic alcohol having 1 to 6 carbon atoms. Preferred mono-hydroxy alcohols include methanol or ethanol.

The alcohol used herein may also be a suitable glycol (e.g., a diol) having up to five carbons. That is, the moiety R¹ may be a hydroxyl-containing alkylene residue having two to five carbon atoms; the foregoing alkylene residue may straight or branched chain. A “hydroxyl-containing alkylene residue” refers to a substructure for R¹ that provides a glycol (e.g., a diol). Preferred glycols for use herein include those in which R¹ is HOCH₂CH₂— (ethylene glycol), HOCH₂CH₂CH₂— or HOCH(CH₃)CH₂— (1,3- or 1,2-propylene glycol), or HO—CH₂C(CH₃)₂CH₂— (neopentyl glycol). Further, when a glycol is employed, the two R¹ groups on the resulting acetal together form a ring structure having from five to eight atoms. That is, a glycol employed herein will form a corresponding alkylene link of two to five carbons between the oxygens of the acetal.

The alcohol should be employed in a molar excess of between about 3 and about 30 with respect to the enol ester. A molar excess of about 5 to about 20 of alcohol compared to the enol ester would be preferred, with a range of about 5 to about 15 being more preferred. The alcohol used in this step may also function as a solvent for this reaction; an inert solvent, such as described above for the first step of the process, may also be employed. In a preferred embodiment, methanol or ethanol is used in excess and functions as both reactant and solvent. The skilled artisan will understand that glycols mentioned above, such as ethylene glycol, 1,2- or 1,3-propylene glycol, or neopentyl glycol, likewise may be employed in excess to function as both solvent and reactant for the production of the acetal.

The catalyst used in the second step of the invention may be chosen from a wide range of mineral or organic acids. Thus, the second step may employ, for example, sulfuric acid, hydrochloric acid, hydrobromic acid, p-toluenesulfonic acid, methanesulfonic acid, camphorsulfonic acid, acetic acid, propionic acid or other similar acids. Among the mineral or organic acids, hydrochloric acid, sulfuric acid or p-toluenesulfonic acid are preferred. The catalyst should be used at a level of between about 0.01 and about 25 weight percent with respect to the enol ester used in the reaction, with the preferred range being between about 0.5 and about 3 weight percent. In the alternative, it may be useful to employ an insoluble, acidic ion-exchange resin as a catalyst; examples include Amberlyst 15 and similar materials such as Amberlite IR-120, Amberlite IRA-400, or a Nafion resin. When an ion exchange resin is used in this step as a catalyst, it may be preferred to use as much as about 5 to about 20 weight %, and preferably about 10 to about 15 weight %, based on the amount of enol ester.

During the reaction of the second step of the invention, in which the enol ester is contacted with an alcohol to produce an acetal, the temperature is at or about the reflux temperature of the alcohol used in the second step. When a high boiling glycol is used (e.g., ethylene glycol), a co-solvent such as toluene may be used in which case the reaction would be advantageously performed at the reflux temperature of the co-solvent. Thus, the second step of the instant invention is typically performed at about 50° to about 120° C., and would preferably be performed at about 55° to about 85° C. The second step, like the first step, is typically run at about atmospheric or about ambient pressure.

Those of skill in the art will recognize that it is advantageous to perform the second step in an anhydrous manner to avoid potential yield loss. Thus, one may employ any of a number of widely available dehydrating agent to scavenge any water that may be present in the system from, for example, the alcohol employed in the second step. Examples suitable dehydrating agents include 2-methoxypropene, trimethyl orthoformate or triethyl orthoformate.

The isolation of product from either step in the inventive reaction sequence may be accomplished by using techniques of preparative organic chemistry known to those of skill in the art, such as distillation under atmospheric or reduced pressure. In addition, while the intermediate enol ester may be isolated and purified, it need not be and may be converted on to the final acetal product without intermediate isolation and purification. Further, the process may be carried out in either a batch or continuous mode of operation.

This invention can be further illustrated by the following examples, although it will be understood that these examples are included merely for purposes of illustration and are not intended to limit the scope of the invention unless otherwise specifically indicated.

### EXAMPLES

### Example 1

Preparation of 4-chloro-1-acetoxy-1-butene: A solution of 62.4 grams (0.80 mole) of acetyl chloride in 750 ml of ethyl acetate was stirred at room temperature under nitrogen atmosphere. There was added in one portion 2.0 grams (0.015 mole, 1.8 mole per cent) of anhydrous zinc chloride. There was then added 56 grams (0.80 mol) of cyclopropane carboxaldehyde at such a rate that the temperature of the reaction mixture remained between 40° and 50° C. during the addition (about 1-2 hours). After completion of the addition, the mixture was stirred at about 40° C. for 5 hours. The mixture was then cooled to room temperature, washed with aqueous 5% sodium bicarbonate, dried over anhydrous magnesium sulfate, and the solvent removed under reduced pressure to afford 120.6 grams of crude product, an approximately 50/50 mixture of the E and Z isomers of 4-chloro-1-acetoxy-1-butene contaminated with several minor impurities as evidenced by gas chromatographic and NMR analyses.

An identical repetition of this procedure afforded 121.7 grams of crude product. The crude products from the two runs were combined and subjected to distillation through a Vigreaux column at 10-11 mm Hg pressure. There was obtained, at a boiling point (bp) of 75-77° C., a total of 178.2 grams (75% of theory) of product of at least 97 area % purity (GC analysis). NMR analysis indicated the presence of almost equal amounts of the E and Z isomers of 4-chloro-1-acetoxy-1-butene.

### Example 2

Preparation of 4-chlorobutyraldehyde dimethyl acetal: A solution of 72 grams (0.49 mole) of 4-chloro-1-acetoxy-1-butene in 300 ml of anhydrous methanol (240 grams, 7.5 moles) was treated with 15 grams of Amberlyst 15 acidic ion-exchange resin catalyst and stirred under reflux overnight, at which time GC analysis indicted complete conversion of the E and Z isomers of 4-chloro-1-acetoxy-1-butene to a single, higher-boiling product accompanied by several minor impurities. The reaction mixture was filtered to recover the catalyst and the filtrate was treated with 5 grams of solid sodium bicarbonate. Excess methanol was recovered by distillation under reduced pressure to a volume of ca. 70 ml. The liquid was decanted from the undissolved sodium bicarbonate and subjected to distillation through a 6″ Vigreaux column at 25 mm Hg. There was obtained 55.0 grams (74.4% of theory) of colorless product boiling at 83-85° C. NMR analysis confirmed that the distillate was pure 4-chlorobutyraldehyde dimethyl acetal.

### Example 3

Preparation of 4-bromo-1-acetoxy-1-butene: A solution of 56.34 grams of acetyl bromide (0.456 mole) in 350 ml of ethyl acetate was treated with 2.0 grams of zinc chloride, followed by dropwise addition of 32.2 grams (0.46 mole) of cyclopropane carboxaldehyde at 40-45° C. over about 2 hours. The mixture was allowed to stir at 45° C. for about 4 hours, washed with aqueous 5% sodium carbonate solution, dried over sodium sulfate, filtered, and the ethyl acetate recovered by distillation at reduced pressure. The residue was distilled through a 6″ Vigreaux column at 6 mm Hg pressure to give 70.6 grams (79.7% of theory) of product boiling at 80-82°. GC and NMR analyses established that the product was a mixture of E and Z isomers of the title compound in about 95% purity.

### Example 4

Preparation of 4-bromobutyraldehyde dimethyl acetal: A mixture of 13.4 grams (0.070 mole) of 4-bromo-1-acetoxy-1-butene, 150 g (4.8 moles) of methanol, 5 g of 1-methoxypropene (added to ensure anhydrous conditions), and 2.5 grams of Amberlyst 15 was stirred under reflux for 3 hours, at which time gas chromatographic analysis indicated complete conversion to a single major product. Workup and purification as in the case of the analogous chloro compound above afforded 9.7 grams (71% yield) of 4-bromobutyraldehyde dimethyl acetal of bp 64-66° C./6 mm Hg. The structure was confirmed by NMR spectroscopy.

### Example 5

Preparation of 4-chlorobutyraldehyde diethyl acetal: A mixture of 13.7 grams (0.092 mole) of 4-chloro-1-acetoxy-1-butene, 100 ml (1.70 mol) of 95% ethanol, about 5 grams of triethyl orthoformate (added to ensure anhydrous conditions), and 1.40 g of Amberlyst 15 was stirred under reflux for 5 hours, at which time GC analysis indicated completion of the reaction. Workup and distillation as described above afforded 10.82 g (65% yield) of product having a boiling point of 45-50° C. at 1 mm Hg. NMR analysis established that the product was pure 4-chlorobutyraldehyde diethyl acetal.

1. A process for preparing an enol ester of a 4-halo-butyraldehyde of the formula 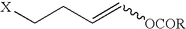
which comprises contacting cyclopropane carboxaldehyde (CPCA) and a carboxylic acid halide having the formula R-COX in the presence of a Lewis acid, wherein R is a branched or straight chain aliphatic hydrocarbon having 1 to 18 carbon atoms, a cyclic hydrocarbon having 3 to 8 carbon atoms, or an aromatic group having 6 to 18 carbon atoms and X is halogen.

2. A process according to claim 1 wherein the Lewis acid is zinc chloride, zinc bromide, zinc iodide, titanium tetrachloride, and titanium tetrabromide.

3. A process according to claim 2 where in R is a straight or branched chain aliphatic hydrocarbon having 1 to 7 carbons and X is chlorine or bromine.

4. A process according to claim 3 wherein the Lewis acid is zinc chloride, R is methyl or ethyl and X is chlorine.

5. A process according to claim 4 wherein the Lewis acid is present at a concentration of about 0.1 to about 10 mole% and the temperature is between about 30° and about 50° C.

6. A process according to claim 1 wherein the process is carried out in the presence of an inert organic solvent.

7. A process for preparing an acetal compound of formula 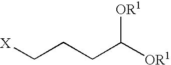
which comprises contacting cyclopropane carboxaldehyde (CPCA) and a carboxylic acid halide having the formula R-COX in the presence of a Lewis acid, to obtain an enol ester of the formula 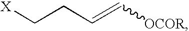
and contacting the enol ester with an alcohol having the formula R¹OH in the presence of an acid, wherein
R is a straight or branched chain aliphatic hydrocarbon having 1 to 18 carbon atoms, a cyclic hydrocarbon having 3 to 8 carbon atoms or an aromatic group having 6 to 18 carbon atoms,
R¹ is a straight or branched chain or cyclic aliphatic hydrocarbon having 1 to 6 carbon atoms, or a straight or branched chain hydroxyl-containing alkylene residue having two to five carbon atoms, and
X is halogen, and
wherein when R¹ in the alcohol is a straight or branched chain hydroxyl-containing alkylene residue having two to five carbon atoms, the two R¹ groups on the acetal together form a ring structure having from five to eight atoms.

8. A process according to claim 7 wherein X is chlorine or bromine, the Lewis acid is zinc chloride, zinc bromide, zinc iodide, titanium tetrachloride, and titanium tetrabromide, and the acid is sulfuric acid, hydrochloric acid, hydrobromic acid, camphorsulfonic acid, p-toluenesulfonic acid, methanesulfonic acid, acetic acid, propionic acid or an acidic ion exchange resin.

9. A process according to claim 8 wherein R is an aliphatic hydrocarbon having 1 to 7 carbons and R¹ is selected from HOCH₂CH₂—, HOCH₂CH₂CH₂—, HOCH(CH₃)CH₂— or HOCH₂C(CH₃)₂CH₂—.

10. A process according to claim 9 wherein the Lewis acid is zinc chloride, R is methyl or ethyl, R¹ is HOCH₂CH₂— or HO—CH₂C(CH₃)₂CH₂—, and X is chlorine.

11. A process according to claim 8 wherein R is an aliphatic hydrocarbon having 1 to 7 carbons and R¹ is methyl or ethyl.

12. A process according to claim 11 wherein the Lewis acid is zinc chloride, the acid is acetic acid, hydrochloric acid, sulfuric acid or p-toluenesulfonic acid, R is methyl or ethyl, and X is chlorine.

13. A process according to claim 8 wherein the Lewis acid is present at a concentration of about 0.1 to about 10 mole % and the temperature is about 30° and about 50° C.

14. A process according to claim 8 wherein the Lewis acid is zinc chloride or zinc bromide, R is methyl or ethyl, R¹ is methyl or ethyl, the acid is an ion exchange resin selected from Amberlyst 15, Amberlite IR-120, Amberlite IRA-400 or a Nafion resin, and X is chlorine.

15. A process according to claim 14 wherein the Lewis acid is zinc chloride and the acid is Amberlyst 15.

16. A process for preparing an acetal compound of formula 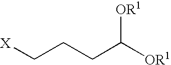
which comprises contacting cyclopropane carboxaldehyde (CPCA) and a carboxylic acid halide having the formula R-COX in the presence of a Lewis acid, to obtain an enol ester of the formula 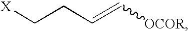
and contacting the enol ester with an alcohol having the formula R¹OH in the presence of an acid, wherein
R¹ is a straight or branched chain aliphatic hydrocarbon having 1 to 7 carbons,
R¹ is a straight or branched chain aliphatic hydrocarbon having 1 to 6 carbon atoms, and
X is chlorine or bromine.

17. A process according to claim 16 wherein the Lewis acid is zinc chloride, zinc bromide, zinc iodide, titanium tetrachloride, or titanium tetrabromide and the acid is sulfuric acid, hydrochloric acid, hydrobromic acid, camphorsulfonic acid, p-toluenesulfonic acid or methanesulfonic acid.

18. A process according to claim 17 wherein the Lewis acid is zinc chloride, the acid is hydrochloric acid, sulfuric acid or p-toluenesulfonic acid, R is methyl or ethyl, R¹ is methyl or ethyl and X is chlorine.

19. A process according to claim 16 wherein the Lewis acid is zinc chloride, zinc bromide, zinc iodide, titanium tetrachloride, and titanium tetrabromide and the acid is an acidic ion exchange resin.

20. A process according to claim 19 wherein the Lewis acid is zinc chloride, the acidic ion exchange resin is Amberlyst 15, Amberlite IR-120, Amberlite IRA-400 or a Nafion resin, R is methyl or ethyl, R¹ is methyl or ethyl and X is chlorine.

21. A process according to claim 20 wherein the acidic ion exchange resin is Amberlyst 15.

22. A process for preparing an acetal compound of formula 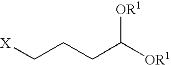
which comprises contacting cyclopropane carboxaldehyde (CPCA) and a carboxylic acid halide having the formula R-COX in the presence of a Lewis acid, to obtain an enol ester of the formula 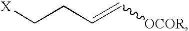
and contacting the enol ester with an alcohol having the formula R¹OH in the presence of an acid, wherein
R is a straight or branched chain aliphatic hydrocarbon having 1 to 7 carbons,
R¹ is a straight or branched chain hydroxyl-containing alkylene residue having two to five carbon atoms,
X is chlorine or bromine, and
the two R¹ groups on the acetal together form a ring structure having from five to eight atoms.

23. A process according to claim 22 wherein the Lewis acid is zinc chloride or zinc bromide, the acid is sulfuric acid, hydrochloric acid or an acidic ion exchange resin, R is methyl or ethyl and R¹ is HOCH₂CH₂— or HO—CH₂C(CH₃)₂CH₂—.
